(19) United States
(12) Patent Application Publication
(10) Pub. No.: US 2026/0081007 A1
(43) Pub. Date: Mar. 19, 2026

(54) TIMELY ALERTS TO PAYERS AND PROVIDERS

(71) Applicants: Pankaj GUPTA, Edison, NJ (US); Saurabh GUPTA, Edison, NJ (US)

(72) Inventors: Pankaj GUPTA, Edison, NJ (US); Saurabh GUPTA, Edison, NJ (US)

(21) Appl. No.: 19/108,012

(22) PCT Filed: Oct. 9, 2023
(86) PCT No.: PCT/US2023/076356

§ 371 Date: Feb. 28, 2025

Related U.S. Application Data
(60) Provisional application No. 63/378,876, filed on Oct. 9, 2022.

(51) Int. Cl. G16H 40/20; G16H 80/00

(52) U.S. Cl. CPC G16H 40/20; G16H 80/00

(57) ABSTRACT

Timely data communications and intelligent functionalities in connection with healthcare events are described. An example computing device or system is configured to detect when a client device of a user arrives at an emergency department (ED) or emergency room (ER). The device is further configured to evaluate alternatives to the ED or ER for the user based on at least one of healthcare options available to the user, health-related data of the user, or geographic locations of alternative providers, among possibly other factors. The device is further configured to push a user notification to the client device. The notification can identify at least one alternative to the ED or ER based on the evaluation of the alternatives to the ED or ER.

### CROSS-REFERENCE TO RELATED APPLICATION

This application claims the benefit of and priority to U.S. Provisional Patent Application No. 63/378,876 , titled “TIMELY ALERTS TO PAYERS AND PROVIDERS,” filed Oct. 9, 2022, the entire contents of which is hereby incorporated by reference herein.

### BACKGROUND

Primary care physicians (PCPs) form the backbone of medical care for many individuals. A long-term relationship with a PCP can keep an individual healthier for longer by maintaining their overall health, treating them when they are sick, catching problems early with screening, and helping them get advanced care when they need it, for example, through referrals to specialists. Therefore, it is essential for individuals to understand the role of a PCP in their healthcare, to prevent medical problems and stay in good health. In addition, with the surge of telehealth capabilities, a PCP is more accessible than ever. Unfortunately, most people wait until they have a problem before finding a medical provider. Delaying preventative care or avoiding the doctor's office altogether may cause an individual to have some unexpected health complications in the future.

### SUMMARY

The present disclosure is directed to systems and methods for timely healthcare data communication and intelligent functionality in connection with a healthcare event such as, for instance, a visit to an emergency department (ED) or an emergency room (ER), by an individual. In particular, various embodiments of the present disclosure are directed to an intelligent healthcare event computing and processing communication framework referred to herein as “Intelo” (or the “Intelo computing framework”) that can be embodied and implemented as a system and methodology. The Intelo computing framework can provide real-time or near-real-time, automated and interactive healthcare data communication and intelligent functionality across various entities in connection with a healthcare event, while the event is occurring or at another time.

Aspects and advantages of embodiments of the present disclosure will be set forth in part in the following description or can be learned from the description or through practice of the embodiments. Other aspects and advantages of embodiments of the present disclosure will become better understood with reference to the appended claims and the accompanying drawings, all of which are incorporated in and constitute a part of this specification. The drawings illustrate example embodiments of the present disclosure and, together with the description, serve to explain the related concepts of the present disclosure.

According to one example embodiment, a computing device or system is configured to detect when a client device of a user arrives at an ED, an ER, or an urgent care (UC) center. The device is further configured to evaluate alternatives to the ED, ER, or UC center for the user based on at least one of healthcare options available to the user, health-related data of the user, or geographic locations of alternative providers, among possibly other factors. The device is further configured to push a user notification to the client device. The notification can identify at least one alternative to the ED, ER, or UC center based on the evaluation of the alternatives to the ED, ER, or UC center.

### BRIEF DESCRIPTION OF THE DRAWINGS

Many aspects of the present disclosure can be better understood with reference to the following figures. The components in the figures are not necessarily to scale, with emphasis instead being placed upon clearly illustrating the concepts of the disclosure. Moreover, repeated use of reference characters or numerals in the figures is intended to represent the same or analogous features, elements, or operations across different figures. Repeated description of such repeated reference characters or numerals is omitted for brevity.

FIG. 1 illustrates a diagram of an example networked environment for timely healthcare data communication and intelligent functionality according to at least one embodiment of the present disclosure.

FIG. 2 illustrates a block diagram of the example networked environment of FIG. 1 according to at least one embodiment of the present disclosure.

FIG. 3 illustrates a flow diagram of an example computer-implemented method for timely healthcare data communication and intelligent functionality according to at least one embodiment of the present disclosure.

FIG. 4 illustrates a flow diagram of another example computer-implemented method for timely healthcare data communication and intelligent functionality according to at least one embodiment of the present disclosure.

### DETAILED DESCRIPTION

An increase in urgent and non-urgent ED care provided to various individuals (e.g., insured, uninsured) has been observed recently. In particular, ED visits have been increasing for conditions that can be treated in a primary care setting by, for instance, a PCP, even though ED visits cost on average more than a visit to a PCP office. Also, many ED visits are potentially avoidable, in part because individuals may be uneducated or undereducated about the healthcare options available to them, including alternatives to an ED, such as an urgent or non-urgent healthcare provider.

As described above, PCPs form the backbone of medical care for many individuals. As such, it is also important for a PCP with whom an individual has an established medical relationship, or a potentially new PCP in some cases, to know in real-time or near-real-time when the individual visits an ED or urgent care center. For instance, it is important for a PCP to have real-time or near-real-time detailed information related to the individual's visit, thereby allowing the PCP to be aware of the visit and intervene in some cases. Generally stated, it is important for a PCP to know in real-time or near-real-time who of their various patients are visiting an ED or urgent care center, when the visits occur and the frequency of such visits, which ED or urgent care centers are visited, where such ED or urgent care centers are located, and why the patients are visiting the ED or urgent care center.

Additionally, proper follow-up care after an urgent or non-urgent ED visit may depend on an individual's PCP being notified in a timely manner, which may or may not occur. In some cases, this may be due in part to the individual's insurance provider not having the patient's current contact information to facilitate the follow-up care for the patient. Also, managed care organizations often require a claim from an ED following an individual's visit, which can take several months in some cases (e.g., 3 months).

The increasing number of ED visits for urgent and non-urgent ED care and delayed or otherwise frustrated follow-up care can cause increased and unnecessary workloads for the personnel, equipment, facilities, computing systems, and other infrastructure of the ED. The unnecessary workloads result in increased inefficiencies and higher overall costs. The increased workloads due to non-urgent ED care may even prevent the ED from treating patients with urgent medical needs in some cases.

Various embodiments of the present disclosure introduce systems, devices, and computer-implemented methods that provide solutions to the above-described problems associated with increasing urgent and non-urgent ED visits and improper follow-up care. The embodiments are directed to timely healthcare data communication and intelligent functionality in connection with healthcare events, such as visits to an ED or an ER, by individuals. More particularly, examples of the present disclosure are directed to the aforementioned Intelo computing framework that can be embodied and implemented as a system and methodology. The Intelo computing framework can provide real-time or near-real-time automated and interactive healthcare data communication and intelligent functionality across various entities in connection with a healthcare event, during and/or after the event.

In one example, the Intelo computing framework can be implemented to identify healthcare options (e.g., providers, treatment options) or alternatives thereof for an individual in connection with a healthcare event, in advance of, during, or after the event. For example, the Intelo computing framework can track the geographic location of a client device of an individual and detect when the client device is within a predefined distance of, has arrived at, or has departed from a healthcare provider such as, for instance, an ED, an ER, or an urgent care (UC) center. In this example, the Intelo computing framework can evaluate alternatives to the ED, ER, or UC center for the individual based on various factors or data described further herein to identify at least one alternative healthcare option for the individual. Based on and/or during such evaluation of alternative options, the Intelo computing framework can also a provide healthcare provider with updated contact information for the individual. The updated contact information can facilitate any needed follow-up care for the individual. Upon detecting the client device has departed from the ED, ER, or UC center, the Intelo computing framework can also be implemented to send a follow-up care notification to at least one of a PCP or an insurance provider, and/or another entity (e.g., family member, friend, caretaker, emergency contact) associated with the individual. The follow-up care notification may at least indicate that the individual has departed the ED, ER, or UC center.

In another example, the Intelo computing framework can be implemented to learn healthcare election patterns of an individual based on at least one of the individual's previous healthcare elections or feedback (e.g., the individual's feedback related to any healthcare provider or treatment option selected or rejected by the individual). In some cases, the Intelo computing framework can be further implemented to provide the individual with recommendations of certain healthcare options or alternatives thereof based on learning such patterns. Such recommendations may be provided in advance of, during, or after the occurrence of a particular healthcare event.

Aspects of the embodiments extend and improve the operations and performance of networked computing systems for timely healthcare data communication and intelligent functionality in connection with a healthcare event such as, for instance, a visit by an individual to an ED or ER. Aspects of the embodiments extend and improve the operations and performance of networked computing systems for automated healthcare event detection (e.g., arrival at an ED or ER), healthcare option and alternatives assessment, and healthcare alternative identification and recommendation. The extension and improvement of the operations of the computing systems can include: (1) improving the performance of the computer systems by identifying an individual's healthcare election patterns in new types of data structures and data metrics; (2) improving the performance of the computer systems through the generation of the new data structures and data metrics; (3) improving the performance of the computer systems in ingesting data from a plurality of different sources to generate the new data structures and assess healthcare election patterns and related metrics with the structures; and so forth.

The embodiments provide other benefits and advantages. For instance, providing timely notifications about one or more urgent or non-urgent ED visits to insurers and physicians can provide opportunities to educate individuals as to available non-ED options. Ready access to information about ED alternatives can provide users with the ability to self-determine their non-ED options. The Intelo computing framework described herein can also enhance user experience by providing users with a means to inform payers (e.g., patients, insurance providers) and providers (e.g., physicians, PCPs, insurance providers) about ED and/or urgent care center visits in real-time. The computing framework can also update insurance and/or healthcare providers, among other parties, about changes to an individual's (e.g., patient's) contact information. The Intelo computing framework also provides payers and providers with the ability to educate an individual about non-ED options, improves timely follow-up, and reduces the costs incurred due to non-critical ED visits.

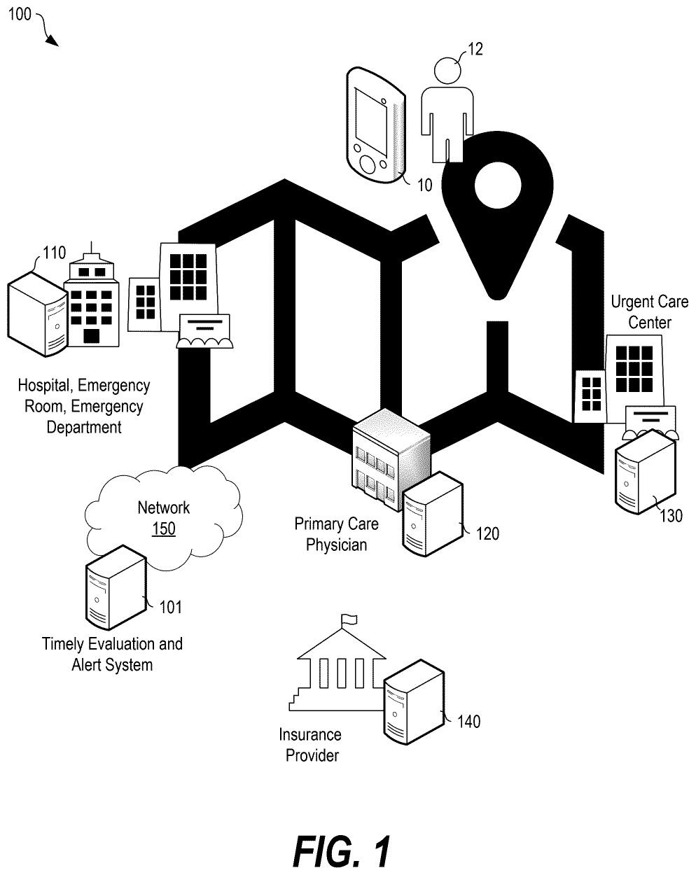

FIG. 1 illustrates a diagram of an example networked environment 100 for timely healthcare data communication and intelligent functionality according to at least one embodiment. In the example shown, the networked environment 100 may be embodied and implemented as an intelligent healthcare event communication network that can facilitate timely (e.g., real-time or near-real-time), automated and interactive, healthcare data communication and intelligent functionality across various entities in connection with a healthcare event, while the event is occurring or at another time. For instance, the networked environment 100 can facilitate various example implementations of the Intelo computing framework described herein.

The networked environment 100 includes a number of computing environments associated with a number of different parties, companies, or organizations. For example, the networked computing environment includes a timely evaluation and alert system 101 (“alert system 101”). The operation and features of the alert system 101 are described in further detail below. The networked environment 100 also includes a computing environment 110 associated with an Emergency Room (ER), Emergency Department (ED), or related treatment center. The networked environment 100 also includes a computing environment 120 associated with a Primary Care Physician (PCP), a computing environment 130 associated with an Urgent Care (UC) center, and a computing environment 140 associated with an insurance provider. The networked environment 100 also includes a number of client devices, such as a client device 10 of a user 12 in FIG. 1.

FIG. 1 is provided as a representative example of an environment in which timely alerts can be provided to payers and providers. In practice, the concepts described herein can be applied using computing systems and environments associated with any number of individuals, service departments, physicians, urgent care centers, insurance providers, and other parties, companies, and organizations. Thus, FIG. 1 is provided as a representative example and is not intended to be limiting.

The alert system 101 and computing environments 110, 120, 130, and 140 can communicate with each other and other devices and systems over a network 150 using any suitable network protocol and communication scheme. Similarly, the client device 10 of the user 12 can communicate with the alert system 101 and one or more of the computing environments 110, 120, 130, and 140 over the network 150 using any suitable network protocol. The network 150 includes, for example, the Internet, intranets, extranets, wide area networks (WANs), local area networks (LANs), wired networks, wireless networks, cable networks, satellite networks, or other suitable networks, etc., or any combination of two or more such networks.

The alert system 101 can be embodied and implemented as a type of computing environment and may include, for example, a server computer or any other system providing computing capability. Alternatively, the alert system 101 may employ a plurality of computing devices that may be arranged, for example, in one or more server banks or computer banks or other arrangements. Such computing devices may be located in a single installation or may be distributed among many different geographical locations. For example, the alert system 101 may include a plurality of computing devices that together may be embodied and implemented as a hosted computing resource, a grid computing resource, and/or any other distributed computing arrangement. In some cases, the alert system 101 may correspond to an elastic computing resource where the allotted capacity of processing, network, storage, or other computing-related resources may vary over time.

Various applications and/or other functionality may be executed at or on the alert system 101 according to various embodiments. Also, various data is stored in a data store accessible to the alert system 101. The data store may be representative of a plurality of data stores as can be appreciated. The data stored in the data store, for example, is at least partly associated with the operation of the various applications and/or functional entities described herein. Each of the computing environments 110, 120, 130, and 140 can be similar to the alert system 101, although each executes different applications or programs for a variety of different purposes.

The components executed on the alert system 101 can include a timely evaluation and alert application (“alert application”), as described herein, among other applications, services, processes, systems, engines, or functionality. The alert application on the alert system 101 can obtain data from a variety of data sources, automatically evaluate certain events and conditions, provide notifications, and perform other functions. The alert system 101 may obtain data from a variety of data sources by way of application programming interfaces (API), for example, direct communication with the computing environment 110, 120, 130, and 140, scraping data from web pages (e.g., using a web crawler or spider application, or a search engine bot), and/or other approaches, and from data previously stored in the networked environment 100 and/or the alert system 101. The alert application on the alert system 101 may also gather data from the client device 10, such as a current position or geolocation of the client device 10, as the user 12 travels from one location to another. The alert application on the alert system 101 can also send notifications and other data to the client device 10 and the computing environment 110, 120, 130, and 140.

The client device 10 is representative of a variety of client devices. The client device 10 can be embodied and implemented as a type of computing system or device and may include, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, smartwatches, head mounted displays, voice interface devices, or other devices. The client device 10 may include a display device such as, for example, one or more liquid crystal display (LCD) displays, gas plasma-based flat panel displays, organic light emitting diode (OLED) displays, electrophoretic ink (E ink) displays, LCD projectors, any combination thereof, or other types of display devices.

The client device 10 may be configured to execute various applications such as a client evaluation and alert application (“client alert application”). The client alert application may be executed in the client device 10, for example, to access network content and notifications served by the alert system 101 and/or other computing environments or systems. To this end, the client alert application may include, for example, a browser, a dedicated application (e.g., “app”), and/or another functional or interactive component to facilitate various operations described in examples herein. The client alert application on the client device 10 may interact with the alert application executing on the alert system 101 to perform various functions including those described below. The client device 10 may be configured to execute applications beyond the client alert application such as, for example, email applications, social networking applications, word processors, spreadsheets, and/or other applications.

Example operations of the client device 10, the alert system 101, and the computing environments 110, 120, 130, and 140 are described herein according to various embodiments. In various examples, the client device 10 and the alert system 101 can execute applications capable of providing timely alerts to, for instance, payers and providers, such as the user 12, the ED, the ER, the PCP, the UC center, the insurance provider, and other parties (e.g., individuals such as a family member, friend, caretaker, or emergency contact of the user 12) and organizations.

In one example, at least one of the client device 10 (e.g., via the client alert application) or the alert system 101 (e.g., via the alert application) can detect or otherwise obtain data indicating that one or more of the client device 10 or the user 12 is within a predetermined distance from, for instance, an ED or ER and/or has arrived at or departed from the ED or ER. This detection can occur via the use of, for instance, the global positioning system (GPS) or other geolocation solutions that can be included in, coupled to, and/or otherwise accessible by either or both of the client device 10 or the alert system 101 for geographic location data and/or GPS functionality. In turn, the alert system 101 (e.g., via the alert application) can provide notifications to the client device 10 or other systems shown in FIG. 1 (e.g., via the client alert application executing on the client device 10 or on such other systems), to notify the user 12 or other parties about one or more alternative healthcare treatment options or providers that may be better suited to the needs of the user 12 (e.g., more effective, less costly). The alert system 101 can provide such notifications when the client device 10 is within a predetermined distance of, arrives at, and/or departs from the ED or ER. The alert system 101 (e.g., via the alert application) can also provide follow-up care notifications when the client device 10 and/or the user 12 departs from the ED or ER. The alert system 101 (e.g., via the alert application) can also circulate or distribute contact information of the user 12 to other parties, such as to the ED, ER, PCP, UC center, or other parties or organizations, when the user 12 arrives at the ED or ER, departs from the ED or ER, or at other times.

In one example, based on a determination that the client device 10 is within a certain distance or proximity to the ED or ER, at least one of the client device 10 or the alert system 101 can evaluate alternatives to the ED or ER for the user 12. For example, a number of treatment options and/or providers may be available to the user 12 for healthcare related services. The options may be more appropriate, cost-effective, timely, and/or otherwise better suited to the needs of the user 12 compared to an ED or ER visit. The options may include the PCP, the UC center, or other options.

The client device 10 (e.g., via the client alert application), the alert system 101 (e.g., via the alert application), or both can perform an evaluation of other options for the user 12 (e.g., alternative treatment facilities or providers, alternative treatment types, alternative physicians for the user 12), based on many different types of data. Examples of such data include, but are not limited to, at least one of healthcare options or data available to the user 12 (e.g., current treatment facilities or providers, treatment types, or physicians available to the user 12), health-related data of the user 12 (e.g., medical history or current medical status of the user 12), geographic locations of alternative PCPs, UC centers, or other providers, the geographic location of the client device 10 and/or the user 12, biometric or physiological data of the user 12 (e.g., obtained via a wearable physiological monitoring device worn by the user 12), and other data. In one example, the evaluation can include searching for and identifying at least one UC center, PCP, or other provider within a predetermined distance from a current geographic location of at least one of the client device 10 or the user 12. In another example, the evaluation can include searching for and identifying at least one UC center, PCP, or other provider within a predetermined distance from a geographic location associated with the user 12 such as, for instance, a home of the user 12, or other location.

Based on the evaluation, at least one of the client device 10 (e.g., via the client alert application) or the alert system 101 (e.g., via the alert application) can also push a user notification to the client device 10. The notification can identify at least one alternative to the ED or ER based on the evaluation. The notification may suggest that the user 12 attend a UC center for care rather than the ED or ER. The notification may additionally or alternatively suggest that the user 12 attend a PCP rather than the ED or ER. The notification can identify at least one of an advantage or a benefit such as, for instance, a cost savings associated with the alternative to the ED or ER. The notification can also identify alternative options that satisfy some predefined evaluation criterion or criteria set by the user 12 using the client alert application on the client device 10 in some cases. For instance, the notification may identify alternative options based on whether they provide certain medical services, based on proximity to the user 12 or the client device 10, based on cost, or some other evaluation criteria of the user 12.

The client device 10 (e.g., via the client alert application) or the alert system 101 (e.g., via the alert application) can also perform other functions described herein with reference to various embodiments and figures. For example, the client device 10 or the alert system 101 can send a provider notification to the computing environment 120 of the PCP, when at least one of the client device 10 or the user 12 arrives at the ED or ER, leaves the ED or ER, or both. The client device 10 or the alert system 101 can also send an insurance provider notification to the computing environment 140 of the insurance provider, when the user 12 arrives at the ED or ER, leaves the ED or ER, or both. The client device 10 or the alert system 101 can also send a provider notification to the computing environment 110 associated with the ED or ER. The client device 10 or the alert system 101 can also provide at least one of the ED or ER, the PCP, or the UC center provider with current or updated contact information of the user 12.

Additionally, when the client device 10 departs from an ED or ER, the client device 10 (e.g., via the client alert application) or the alert system 101 (e.g., via the alert application) can send a follow-up care notification to a PCP of the user 12. The follow-up care notification can identify that the user has departed from the ED or ER. Similarly, when the client device 10 departs from an ED or ER, the client device 10 or the alert system 101 can send a follow-up notification to an insurance provider of the user 12 or other parties. The follow-up notification can identify that the user has departed from the ED or ER.

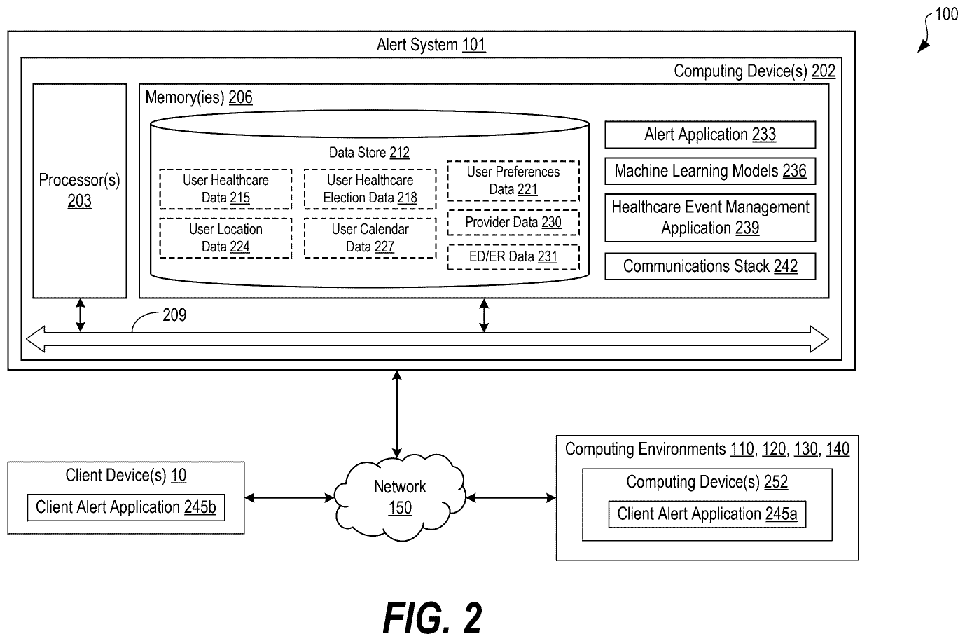

FIG. 2 illustrates a block diagram of the networked environment 100 according to at least one embodiment of the present disclosure. In the example shown, the alert system 101 includes one or more computing devices 202. Further, each of the computing environments 110, 120, 130, and 140 includes one or more computing devices 252 in this example. The alert system 101 and the computing environments 110, 120, 130, and 140 can respectively implement any or all of the computing devices 202 and the computing devices 252 to perform various operations of the Intelo computing framework described in examples herein.

The computing devices 252 may be individually or collectively embodied as the same type of devices as the computing devices 202 of the alert system 101. For instance, the computing devices 252 may individually or collectively include the same structure, components, attributes, and functionality as that of any one of the computing devices 202 or all the computing devices 202. A difference between the computing devices 252 and the computing devices 202 is that any or all of the computing devices 252 can respectively include and execute a client alert application 245a. The client alert application 245a allows the computing environments 110, 120, 130, and 140 to individually communicate and interact with at least one of the alert system 101 or one or more client devices 10. In the example shown, each of the client devices 10 also include a client alert application 245b that allows the client devices 10 to individually communicate and interact with at least one of the alert system 101 or one or more of the computing environments 110, 120, 130, and 140.

Among other types of operations, any of the computing devices 202 can be configured in various examples to provide timely healthcare data communication and intelligent functionality in connection with a healthcare event such as, for instance, a visit by an individual to a healthcare provider such as, for example, an ED or ER. In the example shown, the computing device 202 can provide real-time or near-real-time, automated and interactive healthcare data communication and intelligent functionality across various entities in connection with a healthcare event, while the event is occurring or at another time.

To perform the Intelo computing framework operations described in various examples herein, among other operations, the computing device 202 can include at least one processing and memory system. In the example depicted in FIG. 2, the computing device 202 includes at least one processor 203 and at least one memory 206, both of which are communicatively coupled, operatively coupled, or both, to a local interface 209. In one example, each computing device 202 may be embodied as or include, for example, at least one of a server computing device, a client computing device, a general-purpose computer, a special-purpose computer, a virtual machine, a supercomputer, a quantum computer or processor, a laptop, a tablet, a smartphone, or another type of computing device that can be configured and operable to perform various operations described herein. A detailed description of the computing device 202 and example operations it can perform is provided herein.

The memory 206 includes a data store 212 having user healthcare data 215, user healthcare election data 218, user preferences data 221, user location data 224, user calendar data 227, provider data 230, and ED/ER data 231, among potentially other data, in the example shown. The memory 206 also includes an alert application 233, one or more machine learning models 236, a healthcare event management application 239, a communications stack 242, and potentially other applications. The computing device 202 is coupled to the networks 150 by way of the local interface 209 in this example. In some cases, the computing device 202 can also include other components that are not illustrated in FIG. 2. For instance, an operating system may be stored in the memory 206 and executable by the processor 203. In other examples, one or more components of the computing device 202 shown in FIG. 2 may be omitted.

The processor 203 can be embodied as or include any processing device (e.g., a processor core, a microprocessor, an application specific integrated circuit (ASIC), a field-programmable gate array (FPGA), a controller, a microcontroller, or a quantum processor) and can include one or multiple processors that can be operatively connected. In some examples, the processor 203 can include one or more complex instruction set computing (CISC) microprocessors, one or more reduced instruction set computing (RISC) microprocessors, one or more very long instruction word (VLIW) microprocessors, or one or more processors that are configured to implement other instruction sets.

The memory 206 can be embodied as one or more memory devices and can store data and software or executable-code components executable by the processor 203. The memory 206 can be embodied as, for example, a random-access memory (RAM), read-only memory (ROM), magnetic or other hard disk drive, solid-state, semiconductor, universal serial bus (USB) flash drive, memory card, optical disc (e.g., compact disc (CD) or digital versatile disc (DVD)), floppy disk, magnetic tape, or other types of memory devices.

The memory 206 can store executable-code components associated with the alert application 233, the machine learning models 236, the healthcare event management application 239, the communications stack 242, and the client alert applications 245a, 245b for execution by the processor 203. The memory 206 can also store data such as the data described below that can be stored in the data store 212, among other data. For instance, the memory 206 can also store data indicative of at least one of the user healthcare data 215, the user healthcare election data 218, the user preferences data 221, the user location data 224, the user calendar data 227, the provider data 230, or the ED/ER data 231 described herein, among other data.

The local interface 209 can be embodied as a data bus with an accompanying address/control bus or other addressing, control, and/or command lines. In part, the local interface 209 can be embodied as, for instance, an on-board diagnostics (OBD) bus, a controller area network (CAN) bus, a local interconnect network (LIN) bus, a media oriented systems transport (MOST) bus, ethernet, or another network interface.

The data store 212 can include data for the computing device 202 such as, for instance, one or more unique identifiers for the computing device 202, digital certificates, encryption keys, session keys and session parameters for communications, and other data for reference and processing. The data store 212 can also store computer-readable instructions for execution by the computing device 202 via the processor 203, including instructions for the alert application 233, the machine learning models 236, the healthcare event management application 239, the communications stack 242, and the client alert applications 245a, 245b. In some cases, the data store 212 can also store data indicative of at least one of the user healthcare data 215, the user healthcare election data 218, the user preferences data 221, the user location data 224, the user calendar data 227, the provider data 230, or the ED/ER data 231 described herein, among other data.

The user healthcare data 215 can be indicative of or include, for instance, medical history or current medical status of the user 12. In one example, the user healthcare data 215 can include biometric or physiological data of the user 12. The physiological data may be obtained via a wearable physiological monitoring device worn by the user 12.

The user healthcare election data 218 can be indicative of or include, for instance, historic emergency healthcare options elected by the user 12 (e.g., treatment options or providers previously elected by the user 12 during medical emergencies), historic alternative healthcare options elected by the user 12 (e.g., alternative treatment options or providers previously recommended by the alert application 233 and elected by the user 12), or historic alternative emergency healthcare options elected by the user 12 (e.g., alternative treatment options or providers previously recommended by the alert application 233 during medical emergencies and elected by the user 12). The user healthcare election data 218 can also be indicative of or include historic geographic locations of at least one of historic emergency providers elected by the user 12 (e.g., providers previously elected by the user 12 during medical emergencies), historic alternative providers elected by the user 12 (e.g., alternative providers previously recommended by the alert application 233 and elected by the user 12), or historic alternative emergency providers elected by the user 12 (e.g., alternative providers previously recommended by the alert application 233 during medical emergencies and elected by the user 12).

The user preferences data 221 can be indicative of or include, for instance, evaluation criterion or criteria defined by the user 12 for evaluating alternative healthcare options according to examples herein. For instance, the user 12 can define (e.g., via the client alert application 245b) the user preferences data 221 such that alternative healthcare option evaluations performed by the alert application 233 return alternative healthcare options that, for instance, provide certain medical services, are within a certain proximity of the user 12 or the client device 10, are less than a certain cost, or some other option based on evaluation criteria of the user 12. The user preferences data 221 can further be indicative of or include the profile or contact information of the user 12, as well as any medical related designations defined by the user 12 such as, for instance, an emergency contact name and contact information, and/or what medical information of the user 12 may be accessed by which entities or individuals associated with the user 12, or other user preference data.

The user location data 224 can be indicative of or include, for instance, any or all historic geographic locations of the user 12 and/or the client device 10 as tracked or otherwise obtained by the client device 10, for example, over some period of time. The user location data 224 can further be indicative of or include at least one of a current geographic location or some future (e.g., planned) geographic location or locations of the user 12 and/or the client device 10. The future geographic locations may be obtained or determined from, for instance, at least one of the user calendar data 227 or a mapping application of the computing device 202 having maps of the planned trips of the user 12.

The user calendar data 227 can be indicative of or include, for instance, any planned trips for the user 12. The user calendar data 227 can further be indicative of or include planned healthcare appointments such as, for example, doctor appointments, surgical appointments, and/or other planned medical related services.

The provider data 230 can be indicative of or include, for instance, insurance provider data or data indicative of various terms of a health insurance policy of the user 12 (e.g., user 12 costs for various services and providers under the policy). The provider data 230 may be obtained, for instance, from at least one of the user 12 (e.g., via the client device 10 and the client alert application 245b), an insurance provider (e.g., via a computing device 252 and the client alert application 245a), or another provider.

The ED/ER data 231 can be indicative of or include, for instance, at least one of ER, ED, UC, or PCP data. For example, the ED/ER data 231 can be indicative of or include geographic locations of one or more ERs, EDs, UCs, or PCPs (e.g., previous and/or current locations of ERs, EDs, UCs, or PCPs across, for instance, the United States). The ED/ER data 231 can be obtained (e.g., via the alert application 233) from, for instance, the Centers for Medicare & Medicaid Services (CMS), the Department of Health and Human Services (e.g., which administers Medicare federally, and liaises with state governments to administer Medicaid), or from another data source. The ED/ER data 231 can also be indicative of or include information about what treatment services or medical personnel (e.g., physicians, technicians) are available, in general or at a current time, at various healthcare providers such as, for instance, different EDs, ERs, UCs, PCPs, or other providers.

The alert application 233 can be embodied as one or more software applications or services executing on the computing device 202. The alert application 233 can be executed by the processor 203 of the computing device 202 to perform any or all of the Intelo computing framework operations described in various examples herein.

In one example, the alert application 233 can identify healthcare options (e.g., providers, treatment options) or alternatives thereof for an individual in connection with a healthcare event, in advance of, during, or after the event. For example, the alert application 233 can track the geographic location of the client device 10 of the user 12 and detect when the client device 10 is within a predefined distance of, has arrived at, or has departed from a healthcare provider such as, for instance, an ED or an ER. In this example, the alert application 233 can evaluate alternatives to the ED or ER for the user 12 based on various factors or data described further herein to identify at least one alternative healthcare option for the user 12. Based on and/or during such evaluation of alternative options in this example, the alert application 233 can further provide one or more of the computing environments 110, 120, 130, and 140 with, for instance, updated contact information for the user 12, to facilitate proper follow-up care if needed. Upon detecting the client device 10 has departed from the ED or ER, the alert application 233 can also send a follow-up care notification to at least one of the computing environments 120 or 140, or another entity such as another client device 10 of a family member, guardian, friend, caretaker, emergency contact, among possible others, associated with the user 12. The follow-up care notification may at least indicate that the user 12 has departed the ED or ER.

In another example, the alert application 233 can learn healthcare election patterns of the user 12 based on at least one of the previous healthcare elections or feedback (e.g., the feedback related to any healthcare provider or treatment option selected or rejected by the individual) of the user 12. In one example, the alert application 233 can learn healthcare election patterns of the user 12 based on at least one of historic healthcare options elected by the user 12, health-related data of the user 12, or historic geographic locations of historic providers elected by the user 12.

In one example, the alert application 233 can learn such healthcare election patterns of the user 12 based at least in part on the user healthcare election data 218 and/or any feedback related to such data that has been provided by the user 12 via the client alert application 245b. In another example, the alert application 233 can learn such healthcare election patterns of the user 12 based at least in part on the user healthcare data 215. In yet another example, the alert application 233 can learn at least one of an emergency healthcare election pattern (e.g., indicative of treatment options or providers elected by the user 12 during medical emergencies), an alternative healthcare election pattern (e.g., indicative of alternative treatment options or providers recommended by the alert application 233 and elected by the user 12), or an alternative emergency healthcare election pattern of the user 12 (e.g., indicative of alternative treatment options or providers recommended by the alert application 233 during medical emergencies and elected by the user 12). In various examples, the alert application 233 can employ the machine learning models 236 to learn such healthcare election patterns of the user 12.

In some cases, the alert application 233 can provide the user 12 with recommendations of certain healthcare options currently available to the user 12 (e.g., an ED or ER) or alternatives thereof (e.g., a UC center or PCP) based on learning such healthcare election patterns. The alert application 233 can provide such recommendations in advance of, during, or after the occurrence of a particular healthcare event (e.g., when at least one of the client device 10 or the user 12 is within a predetermined distance of, has arrived at, or has departed from the ED or ER).

The machine learning models 236 can be embodied as one or more software applications or services executing on the computing device 202. The machine learning models 236 can be executed by the processor 203 of the computing device 202 to learn healthcare election patterns of the user 12 and/or provide the user 12 with recommendations of alternative healthcare options based on such patterns.

In automatically learning such healthcare election patterns of the user 12, the alert system 101 and/or the alert application 233 may train and utilize one or more machine learning models 236 such as, for instance, neural networks, convolutional neural networks, deep neural networks, or another machine learning (ML) or artificial intelligence (AI) model. The machine learning models 236 may be trained on a variety of data in order to ascertain patterns in the data through regression analysis. For example, the machine learning models 236 may determine that a certain type of healthcare event such as, for instance, the user 12 or the client device 10 arrival at a certain provider, or a certain geographic location, or a certain type of medical or healthcare emergency, or a certain cost of a treatment may be associated with a certain healthcare election pattern of the user 12. The machine learning models 236 may be continuously or periodically updated based upon new information, thereby further refining and improving the machine learning models 236. For instance, the machine learning models 236 may be updated with data that is continuously or periodically obtained by the alert application 233 from the computing environments 110, 120, 130, and 140. The machine learning models 236 may also be updated with new alternative healthcare options that have been identified and recommended by the alert application 233 to the user 12. The machine learning models 236 may also be updated with any feedback data received from the user 12 via the client alert application 245b in connection with any alternative healthcare option identified and/or recommended by the alert application 233.

The healthcare event management application 239 can be embodied as one or more software applications or services executing on the computing device 202. The healthcare event management application 239 can be executed by the processor 203 of the computing device 202 to provide one or more user interfaces for establishing automated and/or interactive communication and/or intelligent functionality in connection with a healthcare event such as arriving at an ED or ER, among other operations described herein for the Intelo computing framework. The healthcare event management application 239 may be used to establish one or more user interfaces in at least one of the alert application 233, the client alert application 245a, or the client alert application 245b. In one example, any or all of the computing environments 110, 120, 130, and 140 can implement the computing devices 252 to obtain data and insights from an API of the alert application 233, the healthcare event management application 239, and/or the client alert application 245a that allows custom queries and reports for various internal dashboards. In another example, the client device 10 can obtain data and insights from an API of the alert application 233, the healthcare event management application 239, and/or the client alert application 245b that allows custom queries and reports for various internal dashboards. The healthcare event management application 239 can also generate record data that also feeds into and improves the machine learning models 236.

The communications stack 242 can include software and hardware layers to implement data communications such as, for instance, Bluetooth®, Bluetooth® Low Energy (BLE), WiFi®, cellular data communications interfaces, or a combination thereof. Thus, the communications stack 242 can be relied upon by the computing device 202 to establish cellular, Bluetooth®, WiFi®, and other communications channels with the networks 150 and with at least one of the computing devices 252 or the client devices 10.

The communications stack 242 can include the software and hardware to implement Bluetooth®, BLE, and related networking interfaces, which provide for a variety of different network configurations and flexible networking protocols for short-range, low-power wireless communications. The communications stack 242 can also include the software and hardware to implement WiFi® communication, and cellular communication, which also offers a variety of different network configurations and flexible networking protocols for mid-range, long-range, wireless, and cellular communications. The communications stack 242 can also incorporate the software and hardware to implement other communications interfaces, such as X10®, ZigBee®, Z-Wave®, and others. The communications stack 242 can be configured to communicate various data or information amongst the computing device 202, the computing devices 252, and the client devices 10. Examples of such data or information can include, but are not limited to, at least one of the user healthcare data 215, the user location data 224, the provider data 230, or the ED/ER data 231 described herein, among other data.

The networks 150 can include, for instance, the Internet, intranets, extranets, wide area networks (WANs), local area networks (LANs), wired networks, wireless networks (e.g., cellular, WiFi®), cable networks, satellite networks, other suitable networks, or any combinations thereof. The computing devices 202, the computing devices 252, and the client devices 10 can communicate data with one another over the networks 150 using any suitable systems interconnect models and/or protocols. Example interconnect models and protocols include hypertext transfer protocol (HTTP), simple object access protocol (SOAP), representational state transfer (REST), real-time transport protocol (RTP), real-time streaming protocol (RTSP), real-time messaging protocol (RTMP), user datagram protocol (UDP), internet protocol (IP), transmission control protocol (TCP), and/or other protocols for communicating data over the networks 150, without limitation. Although not illustrated, the networks 150 can also include connections to any number of other network hosts, such as website servers, file servers, networked computing resources, databases, data stores, or other network or computing architectures in some cases.

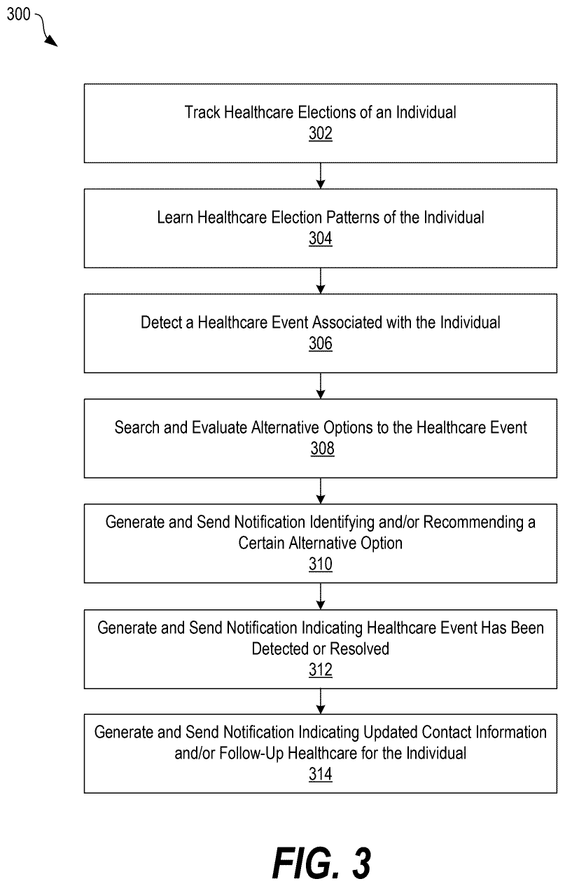

FIG. 3 illustrates a flow diagram of an example computer-implemented method 300 (“method 300”) for timely healthcare data communication and intelligent functionality according to at least one embodiment of the present disclosure. In various examples, the method 300 may be implemented in the context of the networked environment 100 or another environment. In one example, the method 300 can be implemented by at least one of the computing device 202 (e.g., using the alert application 233) or the client device 10 (e.g., the client alert application 245b).

At 302, the method 300 includes tracking healthcare elections of an individual. For example, as described above with reference to FIG. 2, at least one of the computing device 202 (e.g., via the alert application 233) or the client device 10 (e.g., via the client alert application 245b) can track and collect (e.g., record) the user healthcare election data 218 and any feedback data received by either device from the user 12 in connection with any of the user healthcare election data 218. In some examples, the computing device 202 or the client device 10 can track and collect any and all healthcare elections and rejections made by the user 12 with respect to a variety of healthcare options, for instance, over some defined period of time.

In some cases, the computing device 202 or the client device 10 can track and collect healthcare elections and rejections made by the user 12, as well as any feedback received from the user 12 in connection with such choices, with respect to emergency healthcare options during medical emergencies, with respect to alternative healthcare options recommended by the alert application 233 or the client alert application 245b, with respect to alternative emergency healthcare options recommended by the alert application 233 or the client alert application 245b during medical emergencies, or with respect to geographic locations (e.g., historic or current) of at least one of emergency providers during medical emergencies, alternative providers recommended by the alert application 233 or the client alert application 245b, or alternative emergency providers recommended by the alert application 233 or the client alert application 245b during medical emergencies.

At 304, the method 300 includes learning one or more healthcare election patterns of the individual. For example, as described above with reference to FIG. 2, at least one of the computing device 202 (e.g., via the alert application 233) or the client device 10 (e.g., via the client alert application 245b) can implement the machine learning models 236 to learn at least one of an emergency healthcare election pattern, an alternative healthcare election pattern, or an alternative emergency healthcare election pattern of the user 12 based at least in part on the user healthcare election data 218. In some cases, the machine learning models 236 may also rely on any or all of the user healthcare data 215, the user preferences data 221, the user location data 224, the user calendar data 227, the provider data 230, and the ED/ER data 231 to learn such healthcare election patterns of the user 12.

At 306, the method 300 includes detecting a healthcare event associated with the individual. For example, as described above with reference to FIGS. 1 and 2, at least one of the computing device 202 (e.g., via the alert application 233) or the client device 10 (e.g., via the client alert application 245b) can use one or more GPS solutions (e.g., systems, methodologies) to track geographic locations of the user 12. The computing device 202 or the client device 10 can thereby detect when either or both of the client device 10 or the user 12 is within a predetermined distance of, has arrived at, or has departed from a certain healthcare provider (e.g., an ED or ER).

In some examples, being within a predetermined distance from such a provider is an example healthcare event associated with the user 12. In other examples, arrival at such a provider is an example healthcare event associated with the user 12. In still other examples, departure from such a provider is an example healthcare event associated with the user 12. The occurrence of any of such healthcare events can trigger one or more workflows in any or all of the alert application 233, the client alert application 245a, or the client alert application 245b, to cause one or more of the computing device 202, the computing devices 252, or the client device 10 to perform operations of the Intelo computing framework descried in examples herein.

At 308, the method 300 includes searching and evaluating one or more alternative options to the healthcare event. For example, as described above with reference to FIGS. 1 and 2, at least one of the computing device 202 (e.g., via the alert application 233) or the client device 10 (e.g., via the client alert application 245b) can search and evaluate healthcare treatment or provider options that are available to the user 12 as alternatives to those options currently available for the user 12 such as, for instance, an ED or ER.

In some examples, the computing device 202 or the client device 10 can perform such a search and evaluation based on healthcare options or data available to the user 12 (e.g., current treatment facilities or providers, treatment types, or physicians available to the user 12), health-related data of the user 12 (e.g., medical history or current medical status of the user 12), geographic locations of alternative PCPs, UC centers, or other providers, the geographic location of the client device 10 and/or the user 12, biometric or physiological data of the user 12 (e.g., obtained via a wearable physiological monitoring device worn by the user 12), and other data. In other examples, the computing device 202 or the client device 10 can perform such a search and evaluation based at least in part on the above-described healthcare election patterns of the user 12 learned at 304 of the method 300.

At 310, the method 300 includes generating and sending one or more notifications (e.g., user notifications) identifying and/or recommending a certain alternative option for the individual. For example, as described above with reference to FIGS. 1 and 2, at least one of the computing device 202 (e.g., via the alert application 233) or the client device 10 (e.g., via the client alert application 245b) can send one or more of such notifications to at least one of the computing devices 252 or any of the client devices 10 (e.g., the client device 10 associated with the user 12 or another client device 10 associated with family member, guardian, caretaker, friend, emergency contact of the user 12). In one example, such notifications may be sent upon detecting the arrival of either or both of the client device 10 associated with the user 12 or the user 12 at an ED or ER.

At 312, the method 300 includes generating and sending one or more notifications (e.g., insurance or PCP provider notifications) indicating the healthcare event has been detected and/or resolved. For example, as described above with reference to FIGS. 1 and 2, at least one of the computing device 202 (e.g., via the alert application 233) or the client device 10 (e.g., via the client alert application 245b) can send one or more of such notifications to at least one of the computing devices 252 or any of the client devices 10 (e.g., the client device 10 associated with the user 12 or another client device 10 associated with family member, guardian, caretaker, friend, emergency contact of the user 12). In one example, such notifications may be sent upon detecting at least one of the arrival at or departure from an ED or ER by either or both of the client device 10 associated with the user 12 or the user 12.

At 314, the method 300 includes generating and sending one or more notifications (e.g., contact information or follow-up notifications) indicating at least one of updated contact information for the individual or follow-up healthcare information for the individual (e.g., follow-up treatment instructions, appointment reminders). For example, as described above with reference to FIGS. 1 and 2, at least one of the computing device 202 (e.g., via the alert application 233) or the client device 10 (e.g., via the client alert application 245b) can send one or more of such notifications to at least one of the computing devices 252 (e.g., a device of an insurance provider or a PCP of the user 12) or any of the client devices 10 (e.g., the client device 10 associated with the user 12 or another client device 10 associated with family member, guardian, caretaker, friend, emergency contact of the user 12). In one example, such notifications may be sent upon detecting the departure of either or both of the client device 10 associated with the user 12 or the user 12 from an ED or ER.

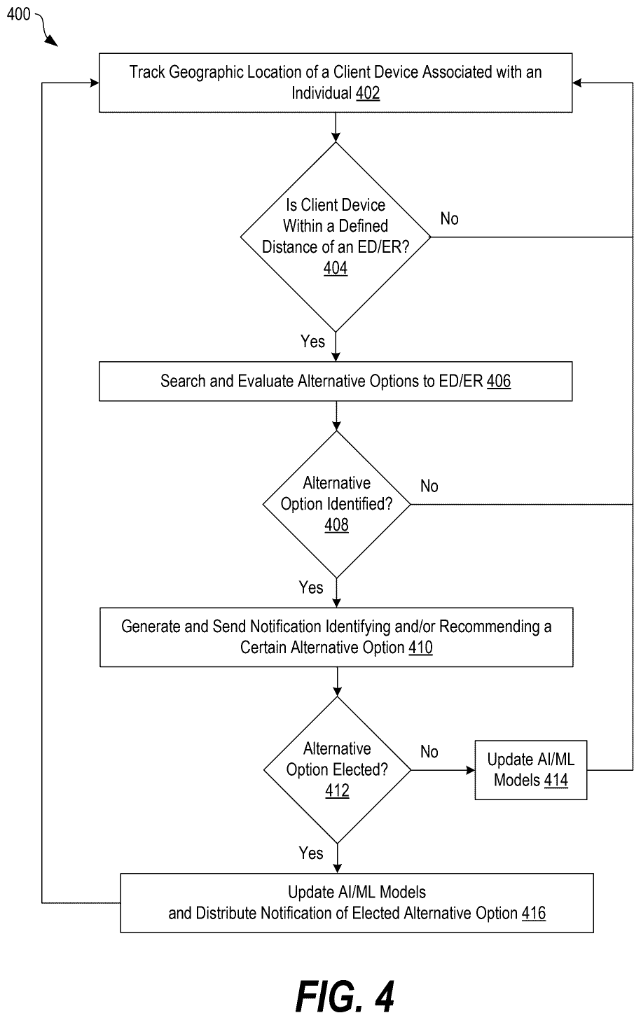

FIG. 4 illustrates a flow diagram of another example computer-implemented method 400 (“method 400”) for timely healthcare data communication and intelligent functionality according to at least one embodiment of the present disclosure.

In various examples, the method 400 may be implemented in the context of the networked environment 100 or another environment. In one example, the method 400 can be implemented by at least one of the computing device 202 (e.g., using the alert application 233) or the client device 10 (e.g., the client alert application 245b).

At blocks 402 and 404, respectively, the method 400 includes tracking geographic locations of a client device associated with an individual and determining whether the client device or the individual is within a defined distance of, for instance, an ED or ER. In one example, at least one of the computing device 202 or the client device 10 can perform the blocks 402 and 404 as described herein with reference to FIGS. 1, 2, and 3. If it is determined at the block 404 that the client device 10 is not within a defined distance of an ED or ER, the method 400 repeats the blocks 402 and 404 until detection of the client device 10 within a defined distance of an ED or ER occurs, at which point the method 400 proceeds to block 406.

At blocks 406 and 408, respectively, the method 400 includes searching and evaluating one or more healthcare options as alternatives to the ED or ER and determining whether any alternative healthcare options are available for the individual in place of or in addition to the ED or ER. In one example, at least one of the computing device 202 or the client device 10 can perform the blocks 406 and 408 as described herein with reference to FIGS. 1, 2, and 3. If it is determined at the block 408 that there are no alternative healthcare options for the user 12, the method 400 repeats the blocks 402 to 408 until alternative healthcare options are identified, at which point the method 400 proceeds to block 410.

At blocks 410 and 412, respectively, the method 400 includes generating and sending one or more notifications identifying and/or recommending one or more particular alternative options for the individual and determining whether any identified or recommended alternative healthcare option was elected or rejected by the individual. In one example, at least one of the computing device 202 or the client device 10 can perform blocks the 410 and 412 as described herein with reference to FIGS. 1, 2, and 3. If it is determined at the block 412 that the user 12 did not elect any identified or recommended alternative healthcare option, the method 400 proceeds to block 414 to update the machine learning models 236 based on the rejection feedback of the user 12. For instance, the computing device 202 or the client device 10 can update, add, or delete various parameters or aspects of any or all of the machine learning models 236 such as, for example, hyperparameters or values thereof, weights or values thereof, layers or input/output configurations thereof, and/or another parameter or aspect of any or all of the machine learning models 236. From the block 414, the method 400 repeats the blocks 402 to 412 until the user 12 elects at least one identified or recommended alternative healthcare option, at which point the method 400 proceeds to block 416.

If it is determined at block 412 that the user 12 did elect at least one identified or recommended alternative healthcare option, at block 416, the method 400 includes updating the machine learning models 236 (e.g., updating any parameter or aspect of any of such models as described above) based on the election feedback of the user 12. From the block 416, the method 400 repeats the blocks 402 to 416. Alternatively, from the block 416, the method 40 may end in some cases.

Referring now to FIG. 2, the memory 206 can store other executable-code components for execution by the processor 203. For example, an operating system can be stored in the memory 206 for execution by the processor 203. Where any component discussed herein is implemented in the form of software, any one of a number of programming languages can be employed such as, for example, C, C++, C#, Objective C, JAVA®, JAVASCRIPT®, Perl, PHP, VISUAL BASIC®, PYTHON®, RUBY, FLASH®, or other programming languages.

As discussed above, the memory 206 can store software for execution by the processor 203. In this respect, the terms “executable” or “for execution” refer to software forms that can ultimately be run or executed by the processor 203, whether in source, object, machine, or other form. Examples of executable programs include, for instance, a compiled program that can be translated into a machine code format and loaded into a random access portion of the memory 206 and executed by the processor 203, source code that can be expressed in an object code format and loaded into a random access portion of the memory 206 and executed by the processor 203, source code that can be interpreted by another executable program to generate instructions in a random access portion of the memory 206 and executed by the processor 203, or other executable programs or code. An executable program can be stored in any portion or component of the memory 206. The memory 206 can be embodied as, for example, a random access memory (RAM), read-only memory (ROM), magnetic or other hard disk drive, solid-state, semiconductor, universal serial bus (USB) flash drive, memory card, optical disc (e.g., compact disc (CD) or digital versatile disc (DVD)), floppy disk, magnetic tape, or other types of memory devices.

In various embodiments, the memory 206 can include both volatile and nonvolatile memory and data storage components. Volatile components are those that do not retain data values upon loss of power. Nonvolatile components are those that retain data upon a loss of power. Thus, the memory 206 can include, for example, a RAM, ROM, magnetic or other hard disk drive, solid-state, semiconductor, or similar drive, USB flash drive, memory card accessed via a memory card reader, floppy disk accessed via an associated floppy disk drive, optical disc accessed via an optical disc drive, magnetic tape accessed via an appropriate tape drive, and/or other memory component, or any combination thereof. In addition, the RAM can include, for example, a static random-access memory (SRAM), dynamic random-access memory (DRAM), or magnetic random-access memory (MRAM), and/or other similar memory device. The ROM can include, for example, a programmable read-only memory (PROM), erasable programmable read-only memory (EPROM), electrically erasable programmable read-only memory (EEPROM), or other similar memory devices.

Also, the processor 203 may represent multiple processors 203 and/or multiple processor cores and the memory 206 may represent multiple memories 206 that operate in parallel processing circuits, respectively. In such a case, the local interface 209 may be an appropriate network that facilitates communication between any two of the multiple processors 203, between any processor 203 and any of the memories 206, or between any two of the memories 206, etc. The local interface 209 may include additional systems designed to coordinate this communication, including, for example, performing load balancing. The processor 203 may be of electrical or of some other available construction.

Any or all of the alert application 233, the machine learning models 236, the healthcare event management application 239, the communications stack 242, and the client alert applications 245a, 245b can be embodied, at least in part, through software or program instructions. The program instructions may be embodied in the form of source code that comprises human-readable statements written in a programming language or machine code that comprises numerical instructions recognizable by a suitable execution system such as a processor 203 in a computer system or other system. The machine code may be converted from the source code, etc. If embodied in hardware, each block may represent a circuit or a number of interconnected circuits to implement the specified logical function(s).

Further, any logic or application described herein, including the alert application 233, the machine learning models 236, the healthcare event management application 239, the communications stack 242, and the client alert applications 245a, 245b, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 202, or in multiple computing devices 202 in the same alert system 101.

As discussed above, the alert application 233, the machine learning models 236, the healthcare event management application 239, the communications stack 242, and the client alert applications 245a, 245b can each be embodied, at least in part, by software or executable-code components for execution by general purpose hardware. Alternatively, the same can be embodied in dedicated hardware or a combination of software, general, specific, and/or dedicated purpose hardware. If embodied in such hardware, each can be implemented as a circuit or state machine, for example, that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components.

Referring now to FIGS. 3 and 4, the flowchart or process diagram shown in each of FIGS. 3 and 4 is representative of certain processes, functionality, and operations of the embodiments discussed herein. Each block can represent one or a combination of steps or executions in a process. Alternatively, or additionally, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as the processor 203. The machine code can be converted from the source code. Further, each block can represent, or be connected with, a circuit or a number of interconnected circuits to implement a certain logical function or process step.

Although the flowchart or process diagram shown in each of FIGS. 3 and 4 illustrates a specific order, it is understood that the order can differ from that which is depicted. For example, an order of execution of two or more blocks can be scrambled relative to the order shown. Also, two or more blocks shown in succession can be executed concurrently or with partial concurrence. Further, in some embodiments, one or more of the blocks can be skipped or omitted. In addition, any number of counters, state variables, warning semaphores, or messages might be added to the logical flow described herein, for purposes of enhanced utility, accounting, performance measurement, or providing troubleshooting aids. Such variations, as understood for implementing the process consistent with the concepts described herein, are within the scope of the embodiments.

Also, any logic or application described herein, including the alert application 233, the machine learning models 236, the healthcare event management application 239, the communications stack 242, and the client alert applications 245a, 245b can be embodied, at least in part, by software or executable-code components and/or stored in any tangible or non-transitory computer-readable medium or device for execution by an instruction execution system such as a general-purpose processor. In this sense, the logic can be embodied as, for example, software or executable-code components that can be fetched from the computer-readable medium and executed by the instruction execution system. Thus, the instruction execution system can be directed by execution of the instructions to perform certain processes such as those illustrated in FIGS. 3 and 4. In the context of the present disclosure, a non-transitory computer-readable medium can be any tangible medium that can contain, store, or maintain any logic, application, software, or executable-code component described herein for use by or in connection with an instruction execution system.

The computer-readable medium can include any physical media such as, for example, magnetic, optical, or semiconductor media. More specific examples of suitable computer-readable media include, but are not limited to, magnetic tapes, magnetic floppy diskettes, magnetic hard drives, memory cards, solid-state drives, USB flash drives, or optical discs. Also, the computer-readable medium can include a RAM including, for example, an SRAM, DRAM, or MRAM. In addition, the computer-readable medium can include a ROM, a PROM, an EPROM, an EEPROM, or other similar memory device.

Disjunctive language, such as the phrase “at least one of X, Y, or Z,” unless specifically stated otherwise, is to be understood with the context as used in general to present that an item, term, or the like, can be either X, Y, or Z, or any combination thereof (e.g., X, Y, and/or Z). Thus, such disjunctive language is not generally intended to, and should not, imply that certain embodiments require at least one of X, at least one of Y, or at least one of Z to be each present. As referenced herein in the context of quantity, the terms “a” or “an” are intended to mean “at least one” and are not intended to imply “one and only one.”

As referred to herein, the terms “include,” “includes,” and “including” are each intended to be inclusive in a manner similar to the term “comprising.” As referenced herein, the terms “or” and “and/or” are generally intended to be inclusive, that is (i.e.), “A or B” or “A and/or B” are each intended to mean “A or B or both.” As referred to herein, the terms “first,” “second,” “third,” and so on, can be used interchangeably to distinguish one component or entity from another and are not intended to signify location, functionality, or importance of the individual components or entities. As referenced herein, the terms “couple,” “couples,” “coupled,” and/or “coupling” refer to chemical coupling (e.g., chemical bonding), communicative coupling, electrical and/or electromagnetic coupling (e.g., capacitive coupling, inductive coupling, direct and/or connected coupling), mechanical coupling, operative coupling, optical coupling, and/or physical coupling.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

Therefore, at least the following is claimed:

1. A computer-implemented method for timely notifications, comprising:
detecting, by at least one computing device, when a client device of a user arrives at an emergency department (ED) or emergency room (ER);
evaluating, by the at least one computing device, alternatives to the ED or ER for the user based on at least one of healthcare options available to the user, health-related data of the user, or geographic locations of alternative providers; and
pushing, by the at least one computing device, a user notification to the client device, the notification identifying at least one alternative to the ED or ER based on the evaluating.

2. The method of claim 1, wherein the detecting comprises geolocating, by the at least one computing device, the client device of the user to within a predetermined distance of the ED or ER.

3. The method of claim 1, wherein:
the evaluating comprises identifying, by the at least one computing device, at least one Urgent Care provider within a predetermined distance from the client device; and
the user notification identifies the Urgent Care provider as an alternative provider to the ED or ER.

4. The method of claim 1, wherein the user notification identifies a Primary Care Physician of the user as an alternative provider to the ED or ER.

5. The method of claim 1, wherein:
the user notification identifies a Primary Care Physician of the user as an alternative provider to the ED or ER; and
the method further comprises sending, by the at least one computing device, a provider notification to a computing environment associated with the Primary Care Physician.

6. The method of claim 1, wherein:
the evaluating comprises identifying, by the at least one computing device, at least one Primary Care Physician within a predetermined distance from the client device or a home address of the user; and
the user notification identifies the Primary Care Physician as an alternative provider to the ED or ER.

7. The method of claim 1, wherein the user notification identifies a cost savings associated with the at least one alternative to the ED or ER.

8. The method of claim 1, further comprising sending, by the at least one computing device, a provider notification to a computing environment associated with the ED or ER based on the evaluating.

9. The method of claim 1, further comprising sending, by the at least one computing device, an insurance provider notification to a computing environment associated with an insurance provider associated with the user.

10. The method of claim 1, further comprising providing, by the at least one computing device and based on a result of the evaluating, at least one of the ED or ER, a Primary Care Physician, or an Urgent Care provider with contact information of the user.

11. The method of claim 1, further comprising:
detecting, by the at least one computing device, when the client device of the user departs from the ED or ER; and
sending, by the at least one computing device, a follow-up care notification to a Primary Care Physician of the user, the follow-up care notification identifying that the user has departed from the ED or ER.

12. The method of claim 1, further comprising:
detecting, by the at least one computing device, when the client device of the user departs from the ED or ER; and
sending, by the at least one computing device, a follow-up notification to an insurance provider of the user, the follow-up notification identifying that the user has departed from the ED or ER.

13. A computing system for timely notifications, comprising:
at least one memory device to store computer-readable instructions thereon; and
at least one processing device configured through execution of the computer-readable instructions to:
detect when a client device of a user arrives at an emergency department (ED) or emergency room (ER);
evaluate alternatives to the ED or ER for the user based on at least one of healthcare options available to the user, health-related data of the user, or geographic locations of alternative providers; and
push a user notification to the client device, the notification identifying at least one alternative to the ED or ER based on the evaluating.

14. The computing system of claim 13, wherein, to detect when the client device of the user arrives at the ED or ER, the at least one processing device is further configured to:
geolocate the client device of the user to within a predetermined distance of the ED or ER.

15. The computing system of claim 13, wherein, to evaluate the alternatives to the ED or ER, the at least one processing device is further configured to:
identify at least one Urgent Care provider within a predetermined distance from the client device; and
the user notification identifies the Urgent Care provider as an alternative provider to the ED or ER.

16. The computing system of claim 13, wherein, to evaluate the alternatives to the ED or ER, the at least one processing device is further configured to:
identify at least one Primary Care Physician within a predetermined distance from the client device or a home address of the user; and
the user notification identifies the Primary Care Physician as an alternative provider to the ED or ER.

17. A computer-implemented method for timely notifications, comprising:
learning, by one or more computing devices, at least one healthcare election pattern of a user based on at least one of historic healthcare options elected by the user, health-related data of the user, or historic geographic locations of historic providers elected by the user; and
pushing a user notification to a client device of the user, the notification recommending at least one alternative healthcare option to a current healthcare option available to the user based on at least one of the healthcare election pattern and a current geographic location of the client device.

18. The method of claim 17, wherein learning the at least one healthcare election pattern comprises:
learning, by the one or more computing devices, at least one of an emergency healthcare election pattern, an alternative healthcare election pattern, or an alternative emergency healthcare election pattern of the user.

19. The method of claim 17, wherein learning the at least one healthcare election pattern comprises:
learning, by the one or more computing devices, the at least one healthcare election pattern of the user based on at least one of historic emergency healthcare options, historic alternative healthcare options, or historic alternative emergency healthcare options elected by the user.

20. The method of claim 17, wherein learning the at least one healthcare election pattern comprises:
learning, by the one or more computing devices, the at least one healthcare election pattern of the user based on historic geographic locations of at least one of historic emergency providers, historic alternative providers, or historic alternative emergency providers elected by the user.
